(19) United States
(12) Patent Application Publication
(10) Pub. No.: US 2019/0167578 A1
(43) Pub. Date: Jun. 6, 2019

(54) Transmucosal Delivery of Terpenes Via Edible Film

(71) Applicant: Daniel Carew Sanders, (US)

(72) Inventor: Daniel Carew Sanders, North Little Rock, AR (US)

(73) Assignee: Daniel Carew Sanders, North Little Rock, AR (US)

(21) Appl. No.: 16/194,107

(22) Filed: Nov. 16, 2018

Related U.S. Application Data
(60) Provisional application No. 62/590,881, filed on Nov. 27, 2017.

(51) Int. Cl. A61K 9/00; A61K 31/015; A61K 31/122; A61K 31/045; A61K 31/121; A61K 31/01; A61K 31/34; A61K 31/05; A61K 31/336; A61K 31/352; A61K 9/70

(52) U.S. Cl. CPC A61K 9/0056; A61K 31/015; A61K 31/122; A61K 31/045; A61K 31/121; A61K 9/7007; A61K 31/34; A61K 31/05; A61K 31/336; A61K 31/352; A61K 31/01

(57) ABSTRACT

The present invention generally optimizes the means and effects of ingesting terpene compounds. As such, the intended effects on the user vary by compound formulation; while the specific process of consumption, and the formulations of the compounds themselves are proprietary. Currently, most of these terpene compounds are known, and consumed in various amounts and combinations as food flavorings; the present invention however, focuses the effects of these compounds to synergize with those of medical cannabis. Furthermore, due to the route of transmucosal administration of these terpene strips, the present invention expedites, optimizes, and elevates both the effects of Medical Cannabis and Industrial Hemp.

### FIELD OF THE INVENTION

The present invention relates to various formulas of terpene and terpenoid compositions from different sources delivered via thin edible film. More particularly, the invention relates to the delivery of these compounds as herbal supplements to boost users' intake of said compound.

### SUMMARY OF EMBODIMENTS

### Cannabis Derived Terpene Embodiment

In one embodiment the invention is directed to administration of supplementary terpenes via dissolving strips, thin-film composites, and transmucosal film. Composition of such a route of administration shall not be limited by the description below, but will pertain to currently recognized thin-film and buccal strip technology. Terpenes may be introduced into product as pure concentrate, a composition of concentrates or microencapsulated compositions of concentrate. Certain embodiments of the invention can contain microencapsulated terpenes, purified terpenes and/or a composition of both. In one embodiment Terpenes may be sourced via cannabis plant matter.

### Extraction Methods/Vendors

Terpenes can be acquired through various means such as CO2 extraction, steam distillation, short path distillation, and any accepted method of isolating and purifying terpenes from cannabis. Consequently, bulk terpenes can be purchased in states that allow medical marijuana; reputable vendors for this, by way of example and not limitation, may be: Entourage Nutritional, Evolabs FreshTerps, and The Terpene Store.

### Non-Cannibis Deprived Terpene Embodiment

In one embodiment the invention is directed to administration of supplementary terpenes via quick dissolve strips, thin-film composites, and transmucosal film. Composition of such route of administration shall not be limited by the description below but will pertain to current recognized thin-film and buccal strip technology. Terpenes may be introduced into product as pure concentrate, a composition of concentrates or microencapsulated compositions of concentrate. Certain embodiments of the invention can contain microencapsulated Terpenes, purified terpenes and/or a composition of both. In one embodiment terpenes will be sourced via non-cannabis matter or wholesale vendors.

### Extraction Methods/Vendors

Due to federal regulations on cannabis-derived products in this embodiment, terpene extracts can be acquired through commercial vendors that specialize in non-cannabis derived products. By way of example and not limitation, possible vendors for our means may be: Emerald or True Terpenes.

### Microencapsulation of Terpenes

As indicated above, various embodiments of the present invention may implement microencapsulation of the terpenes. In general, any terpene maybe microencapsulated. Microencapsulation involves introducing the terpene to a matrix that surrounds its particulate. This is achieved by taking a vector (outer shell), introducing the terpene (inner shell) into it, and then re-sealing the outer shell. The outer shell can commonly be made from yeast cell wall, but this is one non-limiting example. Microencapsulation of the terpenes has various benefits. First, because terpenes are naturally very volatile and are able to boil off the surface in warm temperatures, microencapsulation can be useful for quality control purposes— in particular, microencapsulation can reduce the likelihood of terpene loss during the manufacturing process. When a terpene is microencapsulated it can also be complexed to hide unwanted smells or tastes while still delivering supplement. Microencapsulation may also be useful in modifying absorption rates of the terpenes used. Finally, microencapsulation may help improve the compounding process by creating a powder layer that can be introduced into the non-active layer. The micro encapsulation process may generally be outsourced to a thin film commercial manufacturer.

### BACKGROUND

Terpenes are of a class of hydrocarbons that give many plants and trees their characteristic smell and taste. Many foods and plants, including mangoes, cloves, lemons, thyme, oregano, and roses have terpenes. This unique class of compounds has been isolated and used for many years. In fact, there is even a long history of human terpene consumption. First used in a process termed “aromatherapy” by Rene-Maurice Gattefosse, terpenes were selectively purified to various concentrations, and delivered to aromatherapy participants to inhale the perceivably pleasant scents. This practice has been claimed to help a wide range of physical and mental conditions such as: burns, infections, depression, insomnia, and high blood pressure.

Over the past several years, the increased popularity of cannabis-based products and the growing number of health-conscious consumers have contributed to increased sales in the medical marijuana community. Legalization of medical marijuana in select states has facilitated many new avenues of cannabis treatment. This trend is expected to continue, with sales further increasing in the future. Public opinion on the positive effects of cannabis treatment has also been recently elevated by widely publicized claims of observable positive health benefits. Furthermore, it has recently been discovered that cannabis contains the same terpenes as the above-listed foods and aromatherapy extracts. This accounts for the familiar taste and smell profiles and the huge variation in taste and smell between cannabis strains. Although more than 100 different terpenes have been detected in cannabis there are 11 terpens of interest for our product line with additional used for smell, taste and effect: Limonene, Alpha Bisabolol, Delta 3 Carene, Borneol, Alpha-Pinene, Beta-Pinene, Eucalyptol, Terpineol, Caryophyllene, and Camphene.

Due to federal restrictions on cannabis, no clinical studies have been done on terpene-specific effect and interaction in cannabis treatment to cite at this time; it is, however, commonly reported by patients that various concentrations of different terpenes can have a synergetic effect on cannabinoids when consumed— a phenomenon commonly referred to as the “entourage effect”. With this observation, there appears a need for a quick and effective means of consistently administering terpenes to patients, which becomes a difficult task when current regulations hamper the development of “niche” strains that could help relieve particular symptoms of illnesses. The process of developing niche strains is long and difficult, especially to create specifically desired terpene profiles in plants. Oftentimes it is not until the F8generations that one may see stable plant genetics. Furthermore, current extraction methods of cannabis like rosin pressing, supercritical CO2, and baking edibles can sometimes remove entire terpene profiles from an extract. And due to variation in the growing and curing process between cultivators, terpene profiles can be inconsistent and sometimes entirely non-existent in the same strain of cannabis.

In light of the foregoing reasons there exists an unmet need within the cannabis industry for reliable methods to deliver terpenes which complement the potential medical effects and taste of any smoked plant material.

### BRIEF SUMMARY OF THE INVENTION

The present invention is directed to various oral/buccal transmucosal systems for delivering terpenes to mammal and/or human bodies. Such oral/buccal transmucosal systems include quick dissolve strips, thin-film composites, powders, gels, sprays, tune release lozenge or reservoir packets, and other oral/buccal transmucosal drug/substance delivery systems.

Terpenes can be effectively delivered across the mucous membrane. Transmucosal delivery is particularly attractive because these membranes are very thin and permeable. Such properties allow for the rapid uptake of a substance into the body. This efficient uptake allows substances to bypass some of the body's natural defenses and enhances the therapeutic effect. Transmucosal delivery systems offer several benefits over other methods of delivery including:
Direct Absorption: Absorption through the mucous membrane leads directly to the circulatory system, which allows substances to bypass the gastrointestinal tract as well as first pass liver metabolism (and modification). Anything absorbed via this method is fast acting, with peak blood levels normally in less than 10 minutes (Rapid Onset). This is important for biological therapeutics because a consumer can also ingest 2-10 times less chemical for desired effect in comparison to consuming edibles (Lower Dosages). When users smoke cannabis oftentimes terpenes are rendered unusable by the combustion from a heat source, and whatever terpenes are passed onto the consumer are at significantly smaller amounts than in the whole plant, or than can be delivered via direct absorption.
Rapid Onset: Substances directly enter the circulatory system, which allows the therapeutic to be rapidly transported to the site of need. The faster the substance reached its target area, the faster it can begin to elicit its desired effect.
Lower Dosage: The avoidance of the gastrointestinal tract and first pass metabolism means that much less of the drug can be administered to achieve the same effect as other means of ingestion, allowing for lower dosages to be administered and thus fewer side effects. In some instances during first pass metabolism drugs are also modified before absorption, while transmucosal administration bypasses this mechanism. In some other cases during this metabolization the active ingredients in cannabis are modified to such an extent that the chemicals no longer have an active effect in the body.

Generally speaking, transmucosal drug delivery is generally classified into three systems:
Nasal Transmucosal—Products in this category include nasal sprays, pumps, and gels The majority of the drugs delivered to the nasal passage are anti-inflammatories.
Oral/Buccal Transmucosal—These systems make use of saliva to release the therapeutic. Products include mucoadhesives, quick-dissolve strips or other quick dissolve delivery systems, reservoir pouches or packets, and solid lozenge formulations, and thereby of predominant pertinence to this invention.
Vaginal or Urethral Suppositories—Delivery systems in this category are designated to be absorbed directly by the vaginal of penile capillary beds.

Depending on the system used, a viscous polysaccharide matrix designed to trap foreign particles that may enter the system coats the mouth, nasal passage, or vagina and urethra. This is a defense, which prevents damage to delicate tissues and capillary beds which lie directly underneath the epithelium. Though the mucous membrane protects the body from foreign matter and pathogens, the area is much more permeable than the mucous membrane. This permeability allows drugs delivered to the mucous membrane to quickly enter into circulation and elicit the desired therapeutic effect in a capacity more unaltered by other bodily processes.

In various embodiments, the present invention is directed to a number of transmucosal systems that incorporate terpene compounds or compositions to effectively administer an appropriate dosage of terpene medicament to a user. These various systems are described more fully below.

### DESCRIPTION OF THE CHARTS

Charts 1-3 show examples of possible terpene/terpenoid concentrations of product based off of screen strains via headspace gas chromatography with mass quadrupole spectrophotometer detector. Note these are some of the more common terpenes screened for, and recognized as being orally active.

### DETAILED SUMMARY OF CERTAIN EMBODIMENTS OF THE INVENTION

The present invention relates to various oral/buccal transmucosal systems for delivering terpenes to mammal and/or human bodies. Such oral/buccal transmucosal systems include quick dissolve strips, thin-film composites, powders, gels, sprays, time release lozenge or reservoir packets, and other oral/buccal transmucosal drug/substance delivery systems. The terpenes may be integrated into these delivery systems in a variety of ways, as described in more detail below. In addition, a number of delivery system structures may be used. For example, one oral/buccal transmucosal system that could be used to deliver terpenes is the 3M™ Cydot™ System, offered in several configurations including matrix and reservoir designs. Another oral/buccal transmucosal system that could be used to deliver nutritional supplements is Zengen Inc.'s “oral strip bilayer system”, which is being used in Chloraseptic Relief Strips™. Yet another system could be a “tea bag” device similar to a Skoal Bandit™ product. With various modifications, an oral/buccal transmucosal delivery system similar to these types of systems may be used to administer terpene doses to an individual for treatment of various ailments. The modifications of these systems in order to provide appropriate administration of terpenes is described more completely below.

Terpenes that may be delivered according to the various embodiments of the present invention include but are not limited to Phellandrine, Pulegone, Geraniol, Haxanal, Hexene-1-ol, 2-Heptanone, α-Thujene, α-Pinene, Camphene, 6-Methyl-6hetpen-2-one, β-Pinene, β-Myrcene, Δ-3-Carene, α-Terpinene, Cymene, γ-Terpinene, trans-Sabinene hydrate, cis-Linalool oxide, Linalool, d-Fenchyl alcohol, trans-Pinocarveol, Borneol L, [1,8-Methandien-4-ol], p-Cymen-8-ol, α-Terpineol, Piperitenone, Piperitenone oxide, α-Ylangene, α-Copaene, γ-Caryophyllene, trans-α-Bergamotene, α-Guaiene, trans-β-Famesene, Humulene, Alloaromadendrene, α-Curcumene, β-Selinene, α-Selinene, β-Bisobolene, α-Bulnesene, Selina-3,7(11)-diene, Caryophyllene Oxide, Humulene Oxide, Caryophylla-3,8(13)-dien-5-ol A, Eucalyptol, in addition to various homeopathic/alternative terpenes isolated from cannabis or other matter. Possible terpene compositions are shown, for example, in Chart 1, Chart 2 and Chart 3.

Terpenes/Terpenoids may be used orally to supplement gaps in cannabis strain profiles and/or as general daily supplementation of such chemicals. Furthermore, transmucosal terpenes/terpenoid mixes may be used to modify the taste or perceived effect of smoked plant matter in the body, for a wide range of illnesses and application. In addition, transmucosal delivery of nutrient supplements offers advantages over oral delivery when negative issues are considered with respect to gastrointestinal metabolism, relating to the gastrointestinal tract, the stomach, substance digestion and absorption, swallowing, protocol compliance, and substance effectiveness. The chance of carcinogenic and negative health effects of smoking plant matter to take in terpenes is completely mitigated in all embodiments of invention.

While it is understood that terpene compositions may be integrated into a transmucosal delivery system in a number of ways, the various preferred embodiments of the present invention will now be described. Generally speaking, the present invention includes an edible carrier film (which may be described, for example, as a base film) and an active ingredient (terpene compound or terpene composition) deposited onto or otherwise attached to or in the carrier film, thereby allowing the active ingredient to be administered through the carrier film. One or more additional ingredients may be added in order to achieve various desired results.

With regard to the base film or carrier film, an appropriate edible film carrier for use with various embodiments of the present invention can be selected and created by one of ordinary skill in the art depending upon factors like: the desired rate of dissolution, desired oral feel for the user, the compatibility of the thin film carrier and the active ingredients, production constraints, costs, or other factors. The film can also be of variable thickness, depending upon these same factors. The desired rate for dissolution can vary depending on the specific application for the edible film. For example, for immediate delivery of the active ingredient, the film can be manufactured to rapidly dissolve in the oral cavity, thus delivering the entire dosage of active ingredient at one time. For alternate purposes, the film can also be manufactured to dissolve over an extended period of time, regulating the amount of active ingredient delivered to the oral cavity over a specifically desired length of time.

Specific film formulations and methods of manufacture for these base films or carrier films are known in the art. (For example see U.S. Pat. No. [5,948,430] to ZERBE et al., which incorporated herein by reference. Carrier film formulation usually comprises film formers, bulking agents, softeners, intense artificial sweeteners, sugar alcohol, natural sweeteners, flavors, cooling agents, surfactants, coloring agents, oils, and drying agents. These ingredients are well known and widely available in the food industry.

With regard to the present invention, the primary ingredient for the edible carrier film is the film former, which in most cases can be any water soluble film former. Film formers include but are not limited to: pullulan, guar gum, pectin, xanthan gum, alginates, gelatin, starches (including corn, potato, rice or tapioca), modified starches, maltodextrins, wheat gluten, carboxymethylcellulose, carrageenan konjac or locust bean gum.

In one embodiment, the terpene delivery mechanism of the present invention comprises a bi-layer film delivery system. The bi-layer film consists of one water soluble layer and one dry-coat layer. The second dry-coat layer settles into the first substrate layer, affixing itself thereto. In one embodiment, the dry coat layer is applied to the thin film surface after partial curing of the first (bottom) layer, affixing itself to this bottom layer. In the preferred embodiment, the water soluble layer acts as a substrate layer or carrier layer (but active ingredients may also be dispensed in this layer). Still, while active ingredients (i.e. terpenes or terpene compositions) can be included in either layer, it is preferable that the dry-coat layer contains the active ingredients. Said dry coat layer and similar layers are especially effective with low dose active ingredients that require a very low moisture environment to remain stable. The second layer can also contain substrates and partitioning agents depending on the need or desired effect.

The film is of a size such that it is fast dissolving. The weight per strip may vary. Said weight of the strip may be in the ranges of about 10 to 80 mg, about 20 to 70 mg, about 30 to 60 mg and about 50 mg. The maximum dosing per strip may also vary depending on the choice of active ingredient. While the amount of dosage of a single strip may differ based on various characteristics such as the size of the strip and the formulation used, it is preferred that the maximum dosage is no more than 12.5 mg. Active ingredients can be delivered in a solid or liquid format and depending on dose levels, the active ingredients can be oil or water soluble. Active ingredients that are stable in aqueous systems are preferred. Active ingredients that are not stable in an aqueous system, however, though not preferred, may still be used. Preferably, the dosage per serving is 1-2 strips but may vary depending on the size of the individual strip and other factors known to one skilled in the art.

Individual strips can be made in virtually any size; preferably the strips are 13/16 inch by 1¼ inch rectangles. The thickness of the first layer is preferably in a range between about 0.040 to 1.1 micrometers. The thickness of the second dry coat layer is preferably in the range of about 0.007 to 0.02 micrometers. The thickness of these individual layers may be more or less than the values recited herein, depending on factors known to one skilled in the art, such as load requirements or processing challenges. Any standard manufacturing procedure known in the art may be used to manufacture the film. An example of such a process can be found in U.S. Pat. No. [5,948,430] to ZERBE et al.

Further to the production method described in U.S. Pat. No. [5,948,430] to ZERBE et al., the production of an edible film according to the present invention can also include an aeration step. This step includes aerating the mass prior to application onto a substrate. Aeration is most preferably achieved through mechanical agitation, mechanical reaction, or carbon dioxide aeration. The aeration step produces an edible film having greater thickness and lower density than without aeration.

In one embodiment, the present invention includes an improved composition for delivering a medicament in the oral cavity. Generally speaking, this embodiment utilizes a powder-matrix applied coating and a film layer to administer active ingredients contained in the powder-matrix to the user. Generally speaking, the powder matrix is normally adhered to the film layer to form the applied coating after the film layer has been manufactured. The dry powder matrix will normally contain a minor amount of retained or bound water or other liquid, typically less than about ten percent by weight. The level of moisture in the powder matrix normally should not cause the powder particles to stick or adhere to one another during intermixing of powders to form the powder matrix, and during application of the powder matrix to the filar layer. By way of example and not limitation, the following can be included in the powder matrix as adhesives: poorly water-soluble cellulose derivatives including ethyl cellulose, cellulose acetate and butyl cellulose; shellac; higher fatty acids including stearic acid and palmitic acid.

The film layer is made from any polymer, softener, filler, matrix, or other composition. The film has an acceptable dissolution rate in the oral cavity for a particular thickness of film. For example, if the film has a thickness of 50 microns, it may be desirable for the film to dissolve in the oral cavity within about fifteen seconds. Or it may be desirable for the film to dissolve more slowly. By way of example, and not limitation, the film can be made with pullulan, modified starch, pectin, carrageenan, a maltodextrin, or alginate. By way of example, and not limitation, the film layer can be produced using a highly water-soluble polymer comprising of a natural or synthetic water-soluble polymer. The polymer preferably has good film moldability, produces a soft flexible film, and is safe for human consumption. One such polymer can be a water-soluble cellulose derivative like hydroxypropyl cellulose (HPC), methyl cellulose, hydroxypropyl alkyl cellulose, carboxymethyl cellulose or the salt of carboxymethyl cellulose. Or, the polymer can comprise an acrylic acid copolymer or its sodium, potassium or ammonium salt. The acrylic acid copolymer or its salt can be combined with methacrylic acid, styrene or vinyl type of ether as a comonomer, poly vinyl alcohol, poly vinyl pyrrolidone, polyalkylene glycol, hydroxypropyl starch, alginic acid or its salt, polysaccharide or its derivatives such as tragacanth, bum gelatin, collagen, denatured gelatin, and collagen treated with succinic acid or anhydrous phthalic acid. The following can also, without limitation, be used to produce the film layer pullulan, maltodextrin, pectin, alginates, carrageenan, guar gum, other gelatins, etc.

Also, it should be noted that although terpene substance is typically found and purchased in liquid form, it is the powder state of terpenes that are discussed and addressed at length herein. Thus note that either liquid or powder terpene substance may be used in the composition and manufacture of this invention, to be determined at the discrepancy of one skilled in the art, and during the actual composition/manufacturing process.

The applied coating is a powder matrix including one or more medicaments (i.e. terpenes or terpene compositions, for example). The medicament can be contained in a powder carrier, or can itself be a powder. One advantage of the powder matrix is that it ordinarily does not require the use of a solvent. Another advantage of the powder matrix is that it ordinarily can, if desired, include in addition to the medicament a variety of different auxiliary compositions. A further advantage of the powder matrix is that it can be admixed in a fluidized bed that minimizes the generation of shear and heat. In a fluidized bed, dry air or another gas is dispersed upwardly through a plurality of openings to suspend and intermix the particulate. Any desired means can be used to admix these powders. Another advantage of mixing or suspending powder in a fluidized bed is that the dry air suspending the powder particles tends to prevent agglomeration of the particles. The admixed powder matrix can also be stored (i.e., suspended) in the fluidized bed, prior to the application of the admixed powder matrix to the film layer. The powder matrix can be applied in any desired manner, including but not limited to sifting, screening, atomization, static, mechanical agitation, etc. By way of example and not limitation, the powder matrix can be atomized through a Nordson gun or similar static spray gun using compressed air. Such a gun creates a fine mist spray of powder particles. The gun statically electrically charges the powder particles so they adhere to a surface of the film layer that is receiving the powder particles. Another process for applying the powder particles is to admix the particles with a liquid carrier to form a particle-liquid solution. The particle-liquid solution is sprayed on the film layer. The liquid carrier evaporates, leaving the powder particles on the film. The liquid carrier preferably does not cause the powder particles to dissolve in the liquid carrier.

In addition to the active ingredients, auxiliary compounds may be added to the delivery system in order to achieve various desired results.

For example, one auxiliary composition that can be included in the powder matrix with the medicament is a composition that dissolves slowly over a selected period of time. Such an auxiliary dissolution control composition can be utilized to slow the release of medicament in the oral cavity. Examples of this kind of auxiliary composition are, without limitation, gel forming compositions like carrageenan, gelatin, alginate, pullulan, PVP, and other hydrophilic materials; cyclodextrin, and, inert materials like calcium and fibers. For example, fibers that can be added may comprise of carboxymethylcellulose.

Another auxiliary composition the can be included in the powder matrix with the terpene/terpenoid is an absorption composition that absorbs water or saliva. Such an auxiliary absorption composition can be also be used to slow the release of medicament, and/or to form a gel. The gel can, if desired, cause the strip to become chewable, similar to a very soft jelly-bean. As used herein, an auxiliary composition is termed a gel if, when it is placed in the oral cavity or in contact with another source of bodily liquid, (1) the auxiliary composition absorbs at least four times it weight of water or of saliva or other aqueous solution in a selected period of time, or (2) the auxiliary composition swells to at least three times its thickness in a selected period of time. The selected period of time can vary but preferably is from five seconds to fifteen minutes, most preferably five seconds to five minutes. Examples of gel auxiliary compositions include, without limitation, carboxymethylcellulose, pectin, modified starches, gelatin, and carrageenan. These compositions can be used alone or in combination. One advantage of a gel is that it tends to slow the dissolution of the medicament and to maintain the medicament in the oral cavity for a longer period of time.

A further auxiliary composition that can be included in the powder matrix is a composition that, when placed in the oral cavity in contact with the mucosa therein, adheres to the mucosa. The concentration of such auxiliary adhesion compositions in the powder matrix can be adjusted to vary the length of time that the film adheres to the mucosa, or to vary the adhesive forces generated between the film and mucosa. The auxiliary adhesion compositions adhere to the oral mucosa or to mucosa or tissue in other parts of the body, including the mouth, nose, eyes, vagina, and rectum. Examples of auxiliary adhesion compositions include carboxymethylcellulose, polyvinyl alcohol, polyvinyl pyrrolidone (povidone), sodium alginate, methyl cellulose, hydroxypropyl cellulose, hydroxypropylmethyl cellulose, polyethylene glycols, carbopol, polycarbophil, carhoxyvinyl copolymers, propylene glycol alginate, alginic acid, methyl methacrylate copolymers, tragacanth gum, guar gum, karaya gum, ethylene vinyl acetate, dimethyl polysiloxane, polyoxyalkylene block copolymers, and hydroxyethyl methacrylate copolymers. All examples of composition provided herein are given without limiting the use or inclusion of other comparable or functionally equivalent compositions even though such comparable or functionally equivalent compositions are not listed.

Still another auxiliary composition that can be included in the powder matrix is a flow composition that, when subjected to a curing process, flows to form a smoother or shinier coating on the exterior of the film layer. One preferred curing process is heating the film layer with powder coating to a selected temperature above 76 degrees Fahrenheit to cause the auxiliary flow composition to soften and flow. Examples of this kind of auxiliary composition are lipids (including various animal and vegetable fats), waxes—particularly low melting point waxes, and polyols—particularly low melting point polyols, that can be admixed in powder form or than can included be in powder particles containing a medicament or other compositions. The medicament itself may also have the property of flowing at an elevated temperature in excess of 76 degrees Fahrenheit to form a smoother or shinier coating.

Other auxiliary compositions that can be included in the powder matrix include, without limitation, bulking agents, fillers, pigments (coloring), flavorings, and sweeteners. In various embodiments, combinations of auxiliary compositions can be included in the powder matrix to achieve a desired function. For example, if it is desired to slow the dissolution of a medicament, less soluble fillers and fibers can be included in the powder matrix along with a high concentration of polymers that have a very high degree of ability to adhere to the oral mucosa lining the mouth. Bulking agents that can be included in the powder matrix include, by way of example and not limitation: avicel, sugar alcohols including mannitol and sorbitol and xylitol and isomalt, lactic sugar, sorbitol dextrin, starch, anhydrous calcium phosphate, calcium carbonate, magnesium trisilicate, silica, and amylase.

In various embodiments, the powder matrix can be applied to one or both sides of the film layer. The film layer includes upper outer surface on the top of the film layer and includes a lower outer surface on the bottom of the film. The upper outer surface is generally parallel to the lower outer surface. The top of the film is generally parallel to the bottom of the film. The thickness of the powder matrix layer can vary as desired, but is preferably in the range of 0.001 mm to 3.00 mm, preferably 0.01 mm to 1.00 mm.

If desired, after the powder matrix layer is applied to the film layer, an additional layer or layers can be applied over the powder matrix layer to seal the powder matrix layer, slow the dissolution of the terpene/terpenoid from the powder matrix layer.

If desired, multiple powder matrix layers can be applied to the film layer. The film layer can comprise a laminate of two or more layers. Methods for producing the film layer and incorporating plasticizers, bulking agents, taste modifying agents, pigments, etc. in the film layer are well known in the art and not described in detail herein. Since the medicament is being applied to the film layer in a dry powder form, the likelihood of adverse interactions between the medicament and compositions comprising the film layer is lessened.

In certain embodiments the compositions and films of the present invention may contain at least one flavoring and/or odorant composition that renders the composition or film palatable. Any effective flavor or odor may be used. The flavoring or odor agent or agents are present in any effective amount, including, for example, in an amount ranging from about 0.5 to 40 wt. %, 1 to 30 wt. %, 5 to 15 wt. %, 0.5 to 15 wt. %. The flavorings may be natural or artificial, or combinations thereof.

Unless otherwise specified or required by the context, the term edible as used herein is used interchangeably with the term orally consumable, and generally means that the article may be placed in the mouth, oral cavity, on the tongue, or the like, without significant detrimental effect to the recipient.

Unless otherwise specified or required by the context, the edible films of the present invention may be manufactured in any effective manner. By way of example and not limitation, U.S. patent application Publication Nos. [20010022964], [20020131990] and [20020019447], and U.S. Pat. Nos. [6,419,903], [3,931,146], [5,411,945], [6,010,716], [5,629,003], [5,948,430], [6,177,096], [6,284,264], [5,700,478], [6,449,925], [4,072,551], [4,063,741]—all of which are incorporated herein by reference as if fully set forth herein—describe methods for making edible films. These, and other methods known in the art, or described herein, may be used in accordance with the present invention.

### CHARTS

Chart 1 and Chart 2 detal some possiblene/terpenoid concentrations of product

CHART 2

| Examples of possible | | | | | | |
|---|---|---|---|---|---|---|
| recommended | myr- | caryo- | terpin- | limo- | pi- | linal- |
| compositions | cene | phylene | olene | nene | nene | ool |
| Concentrations in Mg | | | | | | |
| Composition 1 | 8.5 | 2.9 | 0.4 | 1.3 | 5.3 | 0.6 |
| Composition 2 | 3 | 6.6 | 0.1 | 3.6 | 0.4 | 1.5 |
| Composition 3 | 3.6 | 5.9 | 0.1 | 3.9 | 0.6 | 1 |
| Composition 4 | 8.1 | 4.2 | 0.4 | 6.8 | 0.6 | 1.8 |
| Composition 5 | 5.9 | 6 | 0.1 | 3.4 | 5.4 | 0.7 |
| Composition 6 | 4.6 | 1.7 | 0.9 | 1.9 | 0.7 | 0.7 |
| Composition 7 | 6.1 | 2.6 | 0.1 | 2.8 | 1.4 | 1.4 |
| Composition 8 | 1.1 | 1.9 | 0 | 12.9 | 1.7 | 1.1 |
| Composition 9 | 1.5 | 3.2 | 9 | 0.9 | 0.6 | 0.2 |
| Composition 10 | 6.2 | 2.3 | 0.1 | 3.5 | 0.5 | 1 |
| Composition 11 | 3.1 | 1.8 | 0 | 3.7 | 0.6 | 1.3 |
| Composition 12 | 4.7 | 1.5 | 0.1 | 1.3 | 1.5 | 0.2 |
| Composition 13 | 6.7 | 5.8 | 0.2 | 6 | 1.2 | 1.6 |
| Composition 14 | 7.9 | 5 | 0.1 | 6.8 | 0.3 | 1.8 |
| Composition 15 | 3.3 | 3.1 | 4.5 | 0.9 | 0.4 | 0.6 |
| Composition 16 | 7.8 | 4.1 | 0.1 | 0.2 | 2 | 1 |
| Composition 17 | 9.9 | 5.7 | 0.1 | 7.1 | 0.7 | 2.8 |
| Composition 18 | 9.7 | 4.4 | 0.1 | 6.9 | 0.6 | 1.7 |
| Composition 19 | 8.9 | 4.9 | 0.1 | 3.1 | 0.4 | 1.1 |
| Composition 20 | 10.8 | 2.1 | 0.2 | 2.2 | 2.3 | 0.9 |
| Composition 21 | 5.2 | 2.8 | 0.1 | 4.7 | 2.1 | 1.9 |
| Composition 22 | 6.7 | 6.5 | 0 | 1.4 | 0.9 | 1.3 |
| Composition 23 | 4.4 | 5.6 | 0.2 | 3.2 | 1.3 | 1.1 |
| Composition 24 | 3.7 | 3.4 | 0.1 | 5.2 | 0.7 | 1.2 |
| Composition 25 | 2.9 | 2.3 | 6.5 | 1.8 | 0.5 | 0.7 |
| Composition 26 | 7.6 | 1.1 | 0 | 1.7 | 0.3 | 0.3 |
| Composition 27 | 3.6 | 2.5 | 0 | 1.9 | 1.1 | 0.6 |
| Composition 28 | 8.4 | 5.3 | 0.2 | 4.6 | 0.6 | 1.7 |
| Composition 29 | 3.7 | 6.5 | 0.1 | 2.2 | 1.7 | 0.7 |
| Composition 30 | 2 | 4 | 12.5 | 0.8 | 0.8 | 0.1 |
| Composition 31 | 2.5 | 2.9 | 0.1 | 4.1 | 0.4 | 1.9 |
| Composition 32 | 4.5 | 3 | 0.2 | 3.3 | 1.6 | 0.7 |
| Composition 33 | 11.1 | 1.2 | 0.1 | 1 | 6.9 | 0.7 |
| Composition 34 | 16.3 | 5.6 | 0.1 | 1.5 | 1.6 | 0.1 |
| Composition 35 | 7.1 | 1 | 3.7 | 0.7 | 1.9 | 0.4 |
| Composition 36 | 5.4 | 2.2 | 0 | 3.3 | 0.1 | 1 |
| Composition 37 | 2.3 | 5 | 0.8 | 1.1 | 3.6 | 0.3 |
| Composition 38 | 2.3 | 1 | 10.2 | 0.8 | 0.8 | 0.3 |
| Composition 39 | 7.1 | 2.3 | 0.1 | 1.3 | 1.5 | 0.6 |
| Composition 40 | 8.6 | 3 | 0.1 | 6.4 | 3 | 1.7 |
| Composition 41 | 0.7 | 0.2 | 0.3 | 0.1 | 0 | 0.3 |
| Composition 42 | 1 | 0.7 | 0 | 3 | 0 | 0.6 |
| Composition 43 | 6.4 | 4.4 | 0.1 | 6.4 | 2.1 | 0.7 |
| Composition 44 | 4.4 | 4.1 | 0 | 2.9 | 0.9 | 1.1 |
| Composition 45 | 5.5 | 3.4 | 0.2 | 8.8 | 1.1 | 0.3 |
| Composition 46 | 10.3 | 4.4 | 0.1 | 8.5 | 0.6 | 2 |
| Composition 47 | 1.9 | 1.4 | 4.3 | 1.1 | 0.6 | 0.5 |
| Composition 48 | 3 | 0.3 | 0.3 | 0.4 | 0 | 1.4 |
| Composition 49 | 5.3 | 7.3 | 0 | 2 | 0.4 | 0.5 |
| Composition 50 | 6.6 | 1.9 | 0.6 | 1 | 0 | 0.5 |
| Composition 51 | 8.4 | 1.8 | 0 | 0.9 | 2.1 | 0.4 |
| Composition 52 | 10.7 | 3 | 0 | 2 | 2.9 | 0.6 |
| Composition 53 | 3.1 | 1.9 | 0 | 4.3 | 0 | 2.2 |
| Composition 54 | 2.2 | 1.9 | 9 | 1 | 0.7 | 0.5 |
| Composition 55 | 3.4 | 4 | 0 | 2.5 | 0.3 | 1.1 |
| Composition 56 | 1.1 | 2.1 | 3.8 | 0.4 | 0.9 | 0 |
| Composition 57 | 6.1 | 9 | 0.2 | 5 | 0.3 | 0.7 |
| Composition 58 | 3.2 | 3.8 | 0.1 | 5.6 | 0.6 | 2.4 |
| Composition 59 | 13.9 | 1.3 | 0.2 | 0.2 | 1.4 | 0.4 |
| Composition 60 | 2.9 | 1.8 | 0.3 | 1.1 | 0.8 | 0.3 |
| Composition 61 | 11.8 | 1.9 | 2.8 | 1.6 | 2.9 | 0.3 |
| Composition 62 | 6.3 | 4.1 | 0 | 2.5 | 3.3 | 0.9 |
| Composition 63 | 10.1 | 0.3 | 0.3 | 2 | 0 | 1.1 |
| Composition 64 | 1.8 | 1.6 | 0 | 0.5 | 0.3 | 0.4 |
| Composition 65 | 2.8 | 5.2 | 0.2 | 5 | 0.4 | 1.6 |
| Composition 66 | 3.5 | 3.1 | 0.1 | 3.7 | 0.5 | 1.9 |
| Composition 67 | 6 | 5 | 0 | 0 | 2.3 | 1.1 |
| Composition 68 | 2.1 | 1.9 | 12.7 | 1.2 | 0.7 | 0.1 |
| Composition 69 | 4.9 | 1.4 | 0.1 | 2.6 | 0.8 | 0.2 |
| Composition 70 | 5.4 | 4.8 | 0.1 | 2.8 | 0.3 | 0.7 |
| Composition 71 | 3.2 | 2.8 | 2.4 | 2.8 | 0.5 | 0.8 |
| Composition 72 | 6.5 | 4.2 | 0 | 0 | 3 | 0.5 |
| Composition 73 | 2.2 | 1.6 | 0 | 10.2 | 1.3 | 2.3 |
| Composition 74 | 15 | 2.5 | 0 | 0.8 | 0.5 | 0.4 |
| Composition 75 | 2.9 | 1.4 | 12.7 | 2.9 | 0.9 | 0.2 |
| Composition 76 | 8.7 | 1.1 | 0 | 0.7 | 2.7 | 0.3 |
| Composition 77 | 7 | 3.8 | 0 | 1.8 | 1.1 | 0.6 |
| Composition 78 | 1.7 | 3.5 | 0 | 5.8 | 0.4 | 2.2 |
| Composition 79 | 4.2 | 3.3 | 0.1 | 1.4 | 0.7 | 1.4 |
| Composition 80 | 0.2 | 1.2 | 0 | 0.6 | 0.1 | 0.1 |
| Composition 81 | 4.9 | 1.2 | 0 | 1 | 1.4 | 0 |
| Composition 82 | 5.9 | 0.9 | 0.2 | 1.1 | 0.4 | 0.9 |
| Composition 83 | 9.9 | 2.6 | 0.2 | 1.6 | 0 | 0.6 |
| Composition 84 | 1.7 | 2.2 | 8.4 | 1.2 | 0.7 | 0.3 |
| Composition 85 | 5.5 | 5.2 | 5.9 | 0.6 | 2.2 | 0.5 |
| Composition 86 | 1.5 | 4.6 | 0.1 | 2.7 | 0.3 | 1.1 |
| Composition 87 | 5.9 | 2.6 | 0.2 | 5.6 | 0.6 | 0.7 |
| Composition 88 | 1.6 | 1 | 2.8 | 2.5 | 0.5 | 1.3 |
| Composition 89 | 2.9 | 2.6 | 9.9 | 2.6 | 0.9 | 0.3 |

CHART 3
Parameter of compositions that can be used within invention:
May comprise of some or all the following components

| Terpene | Amount of Terpene |
|---|---|
| Phellandrine | 0-2 g |
| Pulegone | 0-2 g |
| Geraniol | 0-2 g |
| Haxanal | 0-2 g |
| Hexene-1-ol | 0-2 g |
| Linalool | 0-2 g |
| α-Thujene | 0-2 g |
| α-Pinene | 0-2 g |
| Camphene | 0-2 g |
| 6-Methyl-6hetpen-2-one | 0-2 g |
| β-Pinene | 0-2 g |
| β-Myrcene | 0-2 g |
| Δ-3-Carene | 0-2 g |
| α-Terpinene | 0-2 g |
| Cymene | 0-2 g |
| ν-Terpinene | 0-2 g |
| trans-Sabinene hydrate | 0-2 g |
| cis-Linalool oxide | 0-2 g |
| Linalool | 0-2 g |
| d-Fenchyl alcohol | 0-2 g |
| trans-Pinocarveol | 0-2 g |
| Borneol L | 0-2 g |
| 1,8-Methandien-4-ol | 0-2 g |
| p-Cymen-8-ol | 0-2 g |
| α-Terpineol | 0-2 g |
| Piperitenone | 0-2 g |
| Piperitenone oxide | 0-2 g |
| α-Ylangene | 0-2 g |
| α-Copaene | 0-2 g |
| y-Caryophyllene | 0-2 g |
| trans-α-Bergamotene | 0-2 g |
| α-Guaiene | 0-2 g |
| trans-β-Farnesene | 0-2 g |
| Humulene | 0-2 g |
| Alloaromadendrene | 0-2 g |
| α-Curcumene | 0-2 g |
| β-Selinene | 0-2 g |
| α-Selinene | 0-2 g |
| β-Bisobolene | 0-2 g |
| α-Bulnesene | 0-2 g |
| Selina-3,7(11)-diene | 0-2 g |
| Caryophyllene Oxide | 0-2 g |
| Humulene Oxide | 0-2 g |
| Caryophylla-3,8(13)-dien-5-ol A | 0-2 g |
| Eucalyptol | 0-2 g |
| Limonene | 0-2 g |
| α-Bisabolol | 0-2 g |

1. A composition comprising:
A. a film layer;
B. a coating, wherein the coating is applied to at least one side of the film layer; and
C. a nutritional supplement.

2. The composition of claim 1, wherein the coating comprises a powder matrix.

3. The composition of claim 1, wherein the film layer comprises terpene/terpenoid composition.

4. The composition of claim 1, wherein the matrix comprises the terpene/terpenoid composition.

5. The composition of claim 4, wherein the terpene/terpenoid is selected from the group consisting of Phellandrine, Pulegone, Geraniol, Haxanal, Hexene-1-ol, 2-Heptanone, α-Thujene, α-Pinene, Camphene, 6-Methyl-6hetpen-2-one, β-Pinene, β-Myrcene, Δ-3-Carene, α-Terpinene, Cymene, γ-Terpinene, trans-Sabinene hydrate, cis-Linalool oxide, Linalool, d-Fenchyl alcohol, trans-Pinocarveol, Borneol L, [1,8-Methandien-4-ol], p-Cymen-8-ol, α-Terpineol, Piperitenone, Piperitenone oxide, α-Ylangene, α-Copaene, y-Caryophyllene, trans-α-Bergamotene, α-Guaiene, trans-β-Farnesene, Humulene, Alloaromadendrene, α-Curcumene, α-Selinene, α-Selinene, β-Bisobolene, α-Bulnesene, Selina-3,7(11)-diene, Caryophyllene Oxide, Humulene Oxide, Caryophylla-3,8(13)-dien-5-ol A, and Eucalyptol.

6. A composition, comprising:
A. a film layer wherein the film layer is configured to rapidly dissolve in an oral cavity; and
B. a coating comprising a terpene/terpenoid composition, wherein the coating is applied to at least one side of the film layer.

7. The composition of claim 6, wherein the film layer dissolves upon being placed in the oral cavity.

8. The composition of claim 6, wherein the nutritional supplement is selected from the group consisting of Phellandrine, Pulegone, Geraniol, Haxanal, Hexene-1-ol, 2-Heptanone, α-Thujene, α-Pinene, Camphene, 6-Methyl-6hetpen-2-one, β-Pinene, β-Myrcene, Δ-3-Carene, α-Terpinene, Cymene, γ-Terpinene, trans-Sabinene hydrate, cis-Linalool oxide, Linalool, d-Fenchyl alcohol, trans-Pinocarveol, Borneol L, [1,8-Methandien-4-ol], p-Cymen-8-ol, α-Terpineol, Piperitenone, Piperitenone oxide, α-Ylangene, α-Copaene, y-Caryophyllene, trans-α-Bergamotene, α-Guaiene, trans-β-Farnesene, Humulene, Alloaromadendrene, α-Curcumene, β-Selinene, α-Selinene, β-Bisobolene, α-Bulnesene, Selina-3,7(11)-diene, Caryophyllene Oxide, Humulene Oxide, Caryophylla-3,8(13)-dien-5-ol A, and Eucalyptolr.

9. The composition of claim 8, wherein the terpene/terpenoid composition is sourced from cannabis.

10. The composition of claim 8, wherein the terpene/terpenoid composition is sourced from non-cannabis sources.
